(19) United States
(12) Patent Application Publication
(10) Pub. No.: US 2022/0395693 A1
(43) Pub. Date: Dec. 15, 2022

(54) SECURITY AND AUTHENTICATION ACCESS FOR MEDICAL IMPLANTS

(71) Applicant: AT&T Intellectual Property I, L.P., Atlanta, GA (US)

(72) Inventor: Joseph Soryal, Glendale, NY (US)

(21) Appl. No.: 17/342,652

(22) Filed: Jun. 9, 2021

(51) Int. Cl. A61N 1/372; G16H 10/60; G16H 40/67

(52) U.S. Cl. CPC A61N 1/37254; G16H 10/60; G16H 40/67

(57) ABSTRACT

A method includes sending a request, wherein the request is for access to bio related information or other information of a biological device or control of the biological device; in response to the first request, receiving a first message, wherein the message comprises an indication of current bio related information for the biological device or historical bio related information for the biological device; and based on the message, authenticating the biological device.

### BACKGROUND

The FDA is warning patients and health care providers that certain implanted medical devices have potential cybersecurity risks. Implantable devices (IDs) are a promising new technology, for example in the medical field IDs may improve the treatment of patients. IDs may include electronic devices implanted in the human body to monitor or treat various sorts of diseases in different organs or to enhance the poor functions of different body parts. There are problems in design from IDs from a cybersecurity perspective. Many IDs when designed did not consider device security as a prominent design goal. As a result, conventional IDs in the market suffer from vulnerabilities, that if exploited can have dramatic consequences.

This background information is provided to reveal information believed by the applicant to be of possible relevance. No admission is necessarily intended, nor should be construed, that any of the preceding information constitutes prior art.

### SUMMARY

The disclosed subject matter provides for securing IDs using blockchain technology. In an example, an apparatus may include a processor and a memory coupled with the processor that effectuates operations. The operations may include receiving bio related information from a biological device; receiving other information; storing the bio related information and the other information; storing statistical information associated with the bio related information or the other information; generating (or otherwise receiving) a threshold for a period associated with the bio related information; sending a first request, wherein the first request is for access to bio related information or the other information of the biological device or control of the biological device; in response to the first request, receiving a first message, wherein the first message comprises a first indication of current bio related information for the biological device or historical bio related information for the biological device; and based on the first message, authenticating the biological device, wherein the authenticating is a based on a comparison of the first indication from the biological device with the threshold.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to limitations that solve any or all disadvantages noted in any part of this disclosure.

### BRIEF DESCRIPTION OF THE DRAWINGS

Reference will now be made to the accompanying drawings, which are not necessarily drawn to scale.

FIG. 1 illustrates an exemplary system to secure a biological device.

FIG. 2 illustrates an exemplary method for security and authentication access for biological devices.

FIG. 3 illustrates an exemplary method for security and authentication access for biological devices.

FIG. 4 illustrates an exemplary method for security and authentication access for biological devices using a crypto-like currency for access or control of a device.

FIG. 5 illustrates a schematic of an exemplary network device.

FIG. 6 illustrates an exemplary communication system that provides wireless telecommunication services over wireless communication networks.

### DETAILED DESCRIPTION

The disclosed subject matter may help secure biological devices (BDs) (e.g., health monitoring devices or health affecting devices). For example, stronger authentication may ensure that commanding device (e.g., a device at a hospital) is a legitimate command device. Health affecting devices may include cardiac defibrillators (CDs) or pacemakers that activate based on a detected health event. Health monitoring devices may include smartwatches that may strictly monitor vitals and pacemakers that monitor and can provide some health affecting action. Health monitoring or health affecting devices (e.g., pacemaker) may be implanted into living things or otherwise attached (e.g., smartwatch) to a living thing.

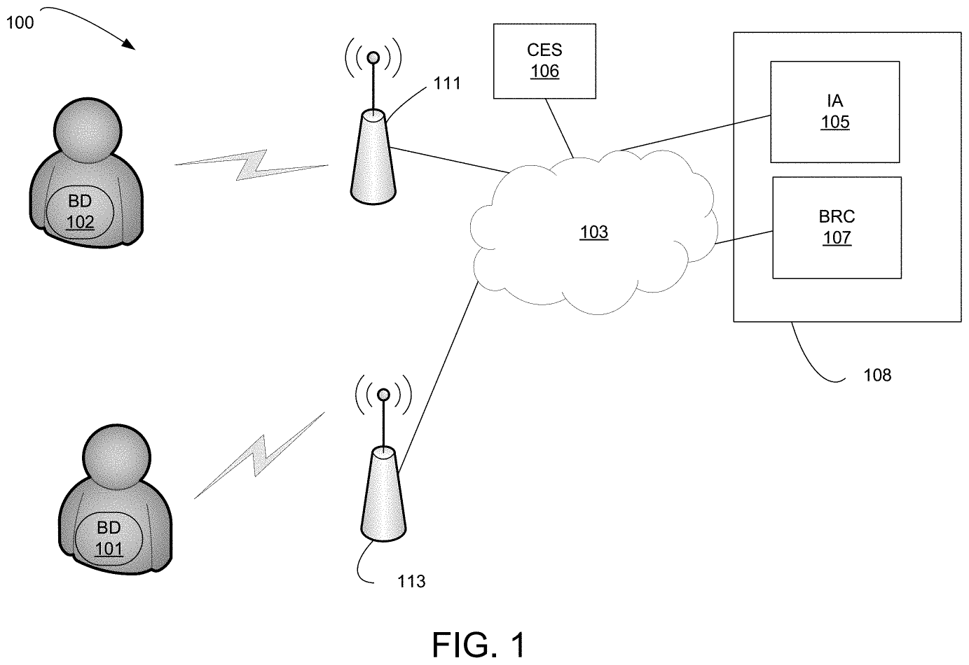

FIG. 1 illustrates an exemplary system to secure biological devices. System 100 may include network 103. BD 101, BD 102, base station 111, base station 113, currency exchange server (CES) 106, implant authenticator (IA) 105, or biometric range collector (BRC) 107 may be communicatively connected with each other via network 103. Network 103 may include vRouters, access points, DNS servers, firewalls, or the like virtual or physical entities. It is contemplated that the functions disclosed herein may be distributed over multiple physical or virtual entities or located within a single physical or virtual entity. In an example, BRC 107 or IA 105 may be functions located within server 108. BD 101 or BD 102 may be able to communicate to network 103 through a wired or wireless connection.

The following information (e.g., imprints) may be captured by BD 101 or devices in proximity to BD 101. The information may include bio related information (e.g., bio imprints), such as heart beat waves pattern, chemical composition of body (e.g., composition of adjacent fluid or tissue), person's voice pattern, person's gait, orientation of BD 101 (e.g., accelerometer or gyroscope information), audio captured, video captured, or sensed temperature, among other things. The information may include location information (e.g., location imprints). The location information may be determined by the consideration of one or more of the following: global positioning system information, wireless signal strength near BD 101, wireless signal presence near BD 101 (e.g., proximate to another ID, such as BD 102 which may be in same or different person), accelerometer information, or gyroscope information, among other things. The information may be recorded over time (e.g., by BD 101).

In an example scenario, BD 101 may record the information (e.g., a particular value or range of values within a period) and send them to a legitimate command and control apparatus (e.g., BRC 107 or IA 105 of server 108) at the beginning of the life cycle of BD 101 inside a patient. BD 101 may request (or receive) from BRC 107 a unique (which may be one-time use) combination of bio imprints before accepting a command from BRC 107. Each ID (e.g., BD 101 vs BD 102) may have a unique random conversion algorithm to convert bio imprints into bits. Physicians or other authorized personnel may have access to override the system, which may be through the use of a specific BRC 107 or BD 102 implanted into the authorized personnel. The disclosed authentication method may be considered an additional verification step (e.g., multi-factor authentication), in addition to conventional access control mechanism (e.g., username and password, device DNA, etc.).

The disclosed subject matter may depend on verifying historical or statistical values not hard identifiers. The BD 101 may take biometrics on regular intervals and send them to BRC 107. BRC 107 may use machine learning to compare the variation of any of the bio stats/readings to similar cases and expected progressions or medication/therapy effects. The expected progressions or medication/therapy effects may be based on just a user associated with BD 101 (e.g., just the users historical information over time) or similarly situated users (e.g., users with similar demographics, ailments, or same type of BD 101). If the recorded bio values are within a threshold range with similar cases around the world, it may help ensure that BD 101 is in the body of the intended patient and not controlled by another malicious entity. Of course, these bio stats/readings may be used to monitor the patient medically but this functionality as disclosed may help ensure BD 101 is still with the correct patient and not manipulated without authorization.

In an example scenario, an authorized physician can manipulate the identifier (ID) remotely via IA 105. The IA 105 may have the physician profiles. IA 105 may query the ID for a collection (two or more) of bio readings (or it may be a range). IA 105 may connect with BRC 107 to retrieve historical readings of BD 101 and the predicted reading at the time IA 105 queried the ID and compares it to the retrieved readings. If the predicted (e.g., expected) readings as determined by the BRC 107 matches the retrieved reading (or are within a threshold range) then access may be allowed. It is contemplated that the bio reading (e.g., biometrics or bio related information), ID, or other information may be restricted so that it is held within a zero-cache buffer or the like.

In addition or alternatively to authentication using bio related information, there may be an access approval based on the use of crypto-like currency for access or control of a device. As shown in FIG. 1, there may be CES 106 which helps manage a crypto currency that may not be related to an actual currency (e.g., monopoly money), but has value in the BD context. CES 106 may determine a crypto currency value needed (e.g., crypto cost) for certain access or control of BD 101. The crypto cost may change regularly in order to add another metric of obscurity to hackers. For example, the crypto cost of action X may be 100 units if BD 101 is at or near a hospital during a first period and the crypto cost may be 2000 units if BD 101 is 10 miles away or the requested change is at a certain period. The bio related information (e.g., an indicated health threatening change to BD 101) may be considered in order to determine the crypto cost. A patient's account may be allocated a first crypto amount (e.g., 600 units), and may need to get additional authorization for a crypto cost that individually (e.g., per action) or cumulatively (e.g., multiple actions) is higher than the first crypto amount. The crypto cost may be set based on any of the following: number of configurations change requested; sensitivity/criticality of requested changes; impact on operation (e.g., patient condition); time of day (e.g., out of normal operation hours would cost more, holidays cost more, etc.); how often this change is required (e.g., less frequently requested feature costs more); how much time, CPU, memory it will take to implement those changes; are the requested changes easily reversable; or several back to back changes may cost more, among other things. The crypto currency mechanism may provide security to the system even if some of the trusted servers are compromised.

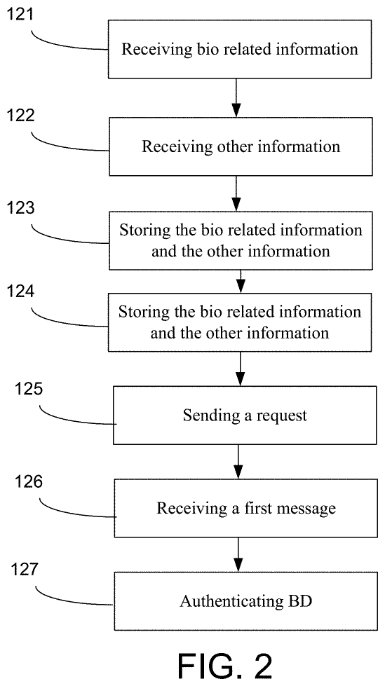

FIG. 2 illustrates an exemplary method for security and authentication access for biological devices. In an example, the below steps may be used for authenticating a biological device.

At step 121, receiving bio related information. Bio related information may include heart beat wave pattern, adjacent (to BD 101) fluid chemical composition, adjacent (to BD 101) tissue chemical composition, fluid pressure, audio of environment surrounding BD 101, video of environment surrounding BD 101, still picture of environment surrounding BD 101, or temperature of environment surrounding BD 101, among other things.

At step 122, receiving other information. For example, BD 101 may be location aware and receive location related information. BD 101 may collect wireless signals nearby (e.g., Wi-Fi, Bluetooth, RAN). The wireless signals may be used to determine location of BD 101. BD 101 may also determine location by using GPS information or the like. Tracking the location of BD 101 may be another factor used to ensure that server 108 is communicating with the correct BD 101 and not other spoofed device or man in the middle attack. BD 101 may record the wireless imprints of wireless devices in the vicinity (e.g., wireless communication type and device identifier for each respective detected device) to establish a route and routine for patient's presence. If an unusual location is detected, then an alert message may be sent about a possible security issue (e.g., BD 101 is illegally cloned, or spoofed). This location and surrounding wireless device imprint may also be used to authenticate server 108 to BD 101. It is contemplated that the location information or bio related information may be used to perform two-way authentication.

At step 123, storing the bio related information and the other information (e.g., location information or wireless imprint information). At step 124, storing statistical information associated with the bio related information or the other information. For example, with reference to step 123 or step 124, information may be stored (e.g., Temp 1 at period A and Temp 2 at period B) or used to create normal thresholds (e.g., an average or median threshold range) of operations for BD 101 for a period. BD 101 may send these ranges to server 108 during the life cycle of BD 101.

At step 125, sending a request, wherein the request is for control of BD 101 (e.g., release insulin) or access to bio related information or the other information of BD 101 (e.g., current insulin level).

At step 126, in response to the request of step 125, receiving a first message, wherein the first message includes an indication of current bio related information for BD 101 or historical bio related information for BD 101.

At step 127, based on the message of step 126, authenticating BD 101. The authenticating may be a based on whether the indication of step 126 from BD 101 reaches a predetermined threshold. As provided herein, a threshold may be generated for a period associated with the bio related information or the statistical computation of the bio related information (e.g., average or median temperature for a particular hour over the course of a month).

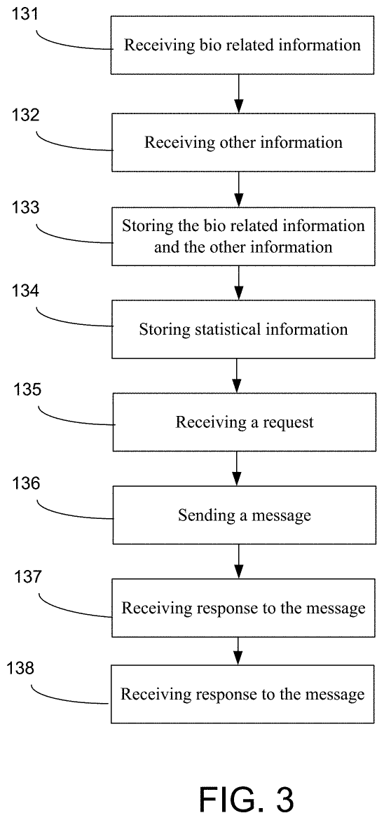

FIG. 3 illustrates an exemplary method for security and authentication access for biological devices. In an example, the below steps may be used for authenticating a remote device communicatively connected with a biological device.

At step 131, receiving bio related information. Bio related information may include heart beat wave pattern, adjacent (to BD 101) fluid chemical composition, adjacent (to BD 101) tissue chemical composition, fluid pressure, audio of environment surrounding BD 101, video of environment surrounding BD 101, still picture of environment surrounding BD 101, or temperature of environment surrounding BD 101, among other things.

At step 132, receiving other information. For example, BD 101 may be location aware and receive location related information. BD 101 may collect wireless signals nearby (e.g., Wi-Fi, Bluetooth, RAN). The wireless signals may be used to determine location of BD 101. BD 101 may also determine location by using GPS information or the like. BD 101 may record the wireless imprints of wireless devices in the vicinity (e.g., wireless communication type and device identifier for each respective detected device) to establish a route and routine for a patient's presence. This location and surrounding wireless device imprint may be used to authenticate server 108 to BD 101. It is contemplated that the location information or bio related information may be used to perform two-way authentication.

At step 133, storing the bio related information and the other information (e.g., location information or wireless imprint information). At step 134, storing statistical information associated with the bio related information or the other information. For example, with reference to step 133 or step 134, information may be stored (e.g., Temp 1 at period A and Temp 2 at period B) or used to create normal thresholds (e.g., an average or median threshold range) of operations for BD 101 for a period. BD 101 may send these ranges to server 108 during the life cycle of BD 101.

At step 135, receiving a request. The request may be from server 108. The request may be for control of BD 101 or access to bio related information or the other information of BD 101.

At step 136, in response to the request of step 135, sending a message. BD 101 may send the message to server 108. The message may include a request for an indication of statistical bio related information for BD 101 or historical bio related information for BD 101.

At step 137, receiving, by BD 101, a response to the message of step 136.

At step 138, based on the response, authenticating the remote device (e.g., server 108). As provided herein, a threshold may be generated for a period associated with the bio related information or the statistical computation of the bio related information (e.g., average or median temperature for a particular hour over the course of a month).

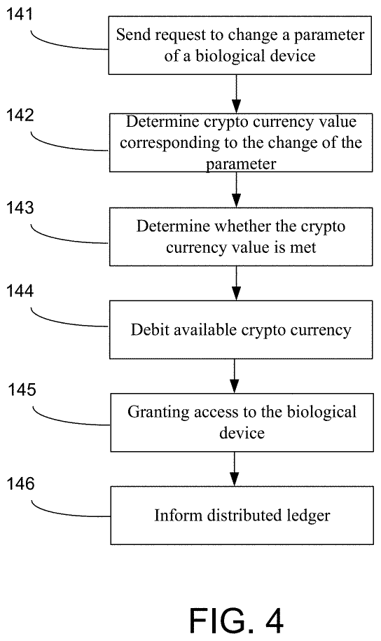

FIG. 4 illustrates an exemplary method for security and authentication access for biological devices using a crypto-like currency for access or control of a device.

At step 141, a request to change a parameter of a biological device may be sent. At step 142, based on the request, the crypto currency value corresponding to the change of the parameter may be determined. At step 143, there may be a determination of whether the crypto currency value corresponding to the change is less than an available crypto currency associated with the biological device. At step 144, based on the crypto currency value corresponding to the change being less than the available crypto currency associated with the biological device, the available crypto currency may be debited. At step 145, in response to the debiting of the available crypto currency, granting access to the biological device and sending an alert indicating the granted access. At step 146, information may be sent about the debit to multiple devices to include in a distributed ledger.

With continued reference to FIG. 4, a request for of a parameter of BD 101 may be received by IA 105. BD 101 (or another device) may determine the crypto currency value to change the parameter. The crypto currency value may be generic units (e.g., 20.45 units). BD 101 may contact CES 106 to obtain the crypto currency value of the 20.45 units in currency with today's rate (as disclosed the rate may change regularly). BD 101 may request IA 105 to remit transmission of 20.45 units worth of currency with the time stamp when BD 101 contacted the CES 106 to get the same rate. IA 105 may contact CES 106 to appropriately convert the crypto currency value. Each message between devices may be cryptographically signed (e.g., unique hash) by CES 106 and then by the IA 105. IA 105 may pay the crypto currency value to the BD 101 and grant access. IA may also verify the hashes using the following: 1) previous knowledge about the hashes; or 2) consult external distributed miners to validate the hashes and values (e.g., distributed ledger mechanism).

A hackers main goal is generally financial gain when accessing vulnerable data. While tampering with an implanted medical device is not an apparent way to make money, the threat still exists. A majority of health record breaches in the first half of 2019 were a result of hacks. Implantable devices are vulnerable to these hacks due to a lack of proper security.

It is contemplated herein that the functions disclosed may operate within one device (e.g., a server) or multiple devices (e.g., multiple servers, base stations, or mobile devices). It is further contemplated that multi-factor authentication may be used, such that interactions from BD 101 to server 108 or server 108 to BD 101 is only successful after presenting two or more pieces of evidence (e.g., bio related information and other information) to an authentication mechanism.

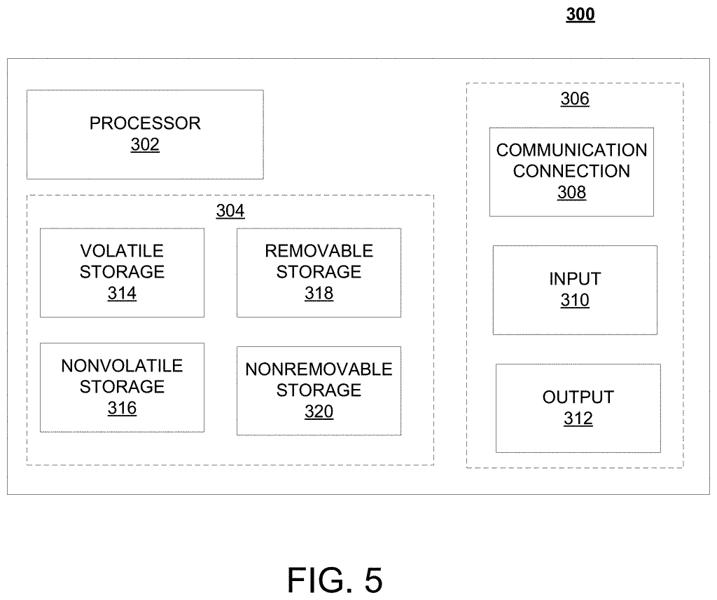

FIG. 5 is a block diagram of network device 300 that may be connected to or comprise a component of cellular network 112 or wireless network 114. Network device 300 may comprise hardware or a combination of hardware and software. The functionality to facilitate telecommunications via a telecommunications network may reside in one or combination of network devices 300. Network device 300 depicted in FIG. 5 may represent or perform functionality of an appropriate network device 300, or combination of network devices 300, such as, for example, a component or various components of a cellular broadcast system wireless network, a processor, a server, a gateway, a node, a mobile switching center (MSC), a short message service center (SMSC), an automatic location function server (ALFS), a gateway mobile location center (GMLC), a radio access network (RAN), a serving mobile location center (SMLC), or the like, or any appropriate combination thereof. It is emphasized that the block diagram depicted in FIG. 5 is exemplary and not intended to imply a limitation to a specific implementation or configuration. Thus, network device 300 may be implemented in a single device or multiple devices (e.g., single server or multiple servers, single gateway or multiple gateways, single controller or multiple controllers). Multiple network entities may be distributed or centrally located. Multiple network entities may communicate wirelessly, via hard wire, or any appropriate combination thereof.

Network device 300 may comprise a processor 302 and a memory 304 coupled to processor 302. Memory 304 may contain executable instructions that, when executed by processor 302, cause processor 302 to effectuate operations associated with mapping wireless signal strength.

In addition to processor 302 and memory 304, network device 300 may include an input/output system 306. Processor 302, memory 304, and input/output system 306 may be coupled together (coupling not shown in FIG. 5) to allow communications between them. Each portion of network device 300 may comprise circuitry for performing functions associated with each respective portion. Thus, each portion may comprise hardware, or a combination of hardware and software. Input/output system 306 may be capable of receiving or providing information from or to a communications device or other network entities configured for telecommunications. For example, input/output system 306 may include a wireless communications (e.g., 3G/4G/GPS) card. Input/output system 306 may be capable of receiving or sending video information, audio information, control information, image information, data, or any combination thereof. Input/output system 306 may be capable of transferring information with network device 300. In various configurations, input/output system 306 may receive or provide information via any appropriate means, such as, for example, optical means (e.g., infrared), electromagnetic means (e.g., RF, Wi-Fi, Bluetooth®, ZigBee®), acoustic means (e.g., speaker, microphone, ultrasonic receiver, ultrasonic transmitter), or a combination thereof. In an example configuration, input/output system 306 may comprise a Wi-Fi finder, a two-way GPS chipset or equivalent, or the like, or a combination thereof.

Input/output system 306 of network device 300 also may contain a communication connection 308 that allows network device 300 to communicate with other devices, network entities, or the like. Communication connection 308 may comprise communication media. Communication media typically embody computer-readable instructions, data structures, program modules or other data in a modulated data signal such as a carrier wave or other transport mechanism and includes any information delivery media. By way of example, and not limitation, communication media may include wired media such as a wired network or direct-wired connection, or wireless media such as acoustic, RF, infrared, or other wireless media. The term computer-readable media as used herein includes both storage media and communication media. Input/output system 306 also may include an input device 310 such as keyboard, mouse, pen, voice input device, or touch input device. Input/output system 306 may also include an output device 312, such as a display, speakers, or a printer.

Processor 302 may be capable of performing functions associated with telecommunications, such as functions for processing broadcast messages, as described herein. For example, processor 302 may be capable of, in conjunction with any other portion of network device 300, determining a type of broadcast message and acting according to the broadcast message type or content, as described herein.

Memory 304 of network device 300 may comprise a storage medium having a concrete, tangible, physical structure. As is known, a signal does not have a concrete, tangible, physical structure. Memory 304, as well as any computer-readable storage medium described herein, is not to be construed as a signal. Memory 304, as well as any computer-readable storage medium described herein, is not to be construed as a transient signal. Memory 304, as well as any computer-readable storage medium described herein, is not to be construed as a propagating signal. Memory 304, as well as any computer-readable storage medium described herein, is to be construed as an article of manufacture.

Memory 304 may store any information utilized in conjunction with telecommunications. Depending upon the exact configuration or type of processor, memory 304 may include a volatile storage 314 (such as some types of RAM), a nonvolatile storage 316 (such as ROM, flash memory), or a combination thereof. Memory 304 may include additional storage (e.g., a removable storage 318 or a non-removable storage 320) including, for example, tape, flash memory, smart cards, CD-ROM, DVD, or other optical storage, magnetic cassettes, magnetic tape, magnetic disk storage or other magnetic storage devices, USB-compatible memory, or any other medium that can be used to store information and that can be accessed by network device 300. Memory 304 may comprise executable instructions that, when executed by processor 302, cause processor 302 to effectuate operations to map signal strengths in an area of interest.

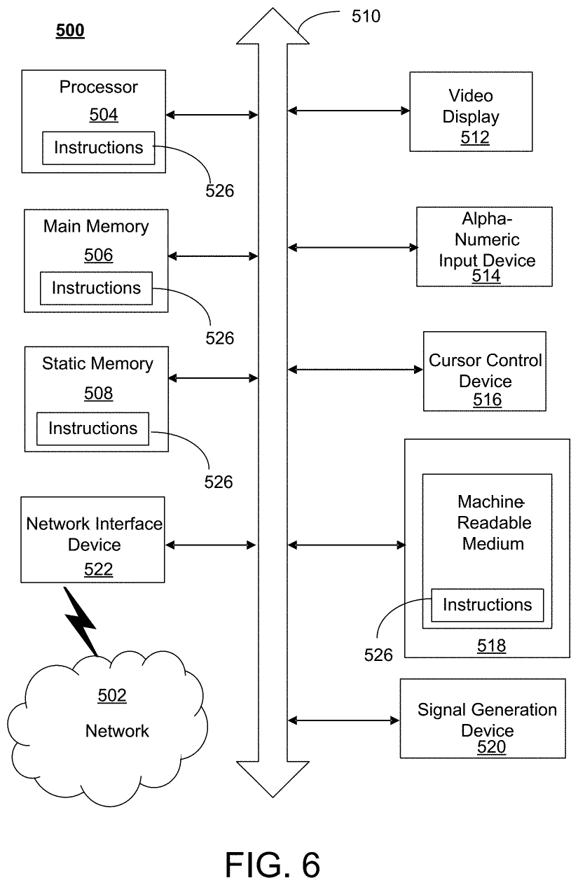

FIG. 6 depicts an exemplary diagrammatic representation of a machine in the form of a computer system 500 within which a set of instructions, when executed, may cause the machine to perform any one or more of the methods described above. One or more instances of the machine can operate, for example, as processor 302, BD 101, BD 102, base station 11, base station 113, CES 106, server 108, and other devices of FIG. 1. In some examples, the machine may be connected (e.g., using a network 502) to other machines. In a networked deployment, the machine may operate in the capacity of a server or a client user machine in a server-client user network environment, or as a peer machine in a peer-to-peer (or distributed) network environment.

The machine may comprise a server computer, a client user computer, a personal computer (PC), a tablet, a smart phone, a laptop computer, a desktop computer, a control system, a network router, switch or bridge, or any machine capable of executing a set of instructions (sequential or otherwise) that specify actions to be taken by that machine. It will be understood that a communication device of the subject disclosure includes broadly any electronic device that provides voice, video or data communication. Further, while a single machine is illustrated, the term “machine” shall also be taken to include any collection of machines that individually or jointly execute a set (or multiple sets) of instructions to perform any one or more of the methods discussed herein.

Computer system 500 may include a processor (or controller) 504 (e.g., a central processing unit (CPU)), a graphics processing unit (GPU, or both), a main memory 506 and a static memory 508, which communicate with each other via a bus 510. The computer system 500 may further include a display unit 512 (e.g., a liquid crystal display (LCD), a flat panel, or a solid state display). Computer system 500 may include an input device 514 (e.g., a keyboard), a cursor control device 516 (e.g., a mouse), a disk drive unit 518, a signal generation device 520 (e.g., a speaker or remote control) and a network interface device 522. In distributed environments, the examples described in the subject disclosure can be adapted to utilize multiple display units 512 controlled by two or more computer systems 500. In this configuration, presentations described by the subject disclosure may in part be shown in a first of display units 512, while the remaining portion is presented in a second of display units 512.

The disk drive unit 518 may include a tangible computer-readable storage medium on which is stored one or more sets of instructions (e.g., software 526) embodying any one or more of the methods or functions described herein, including those methods illustrated above. Instructions 526 may also reside, completely or at least partially, within main memory 506, static memory 508, or within processor 504 during execution thereof by the computer system 500. Main memory 506 and processor 504 also may constitute tangible computer-readable storage media.

As described herein, a telecommunications system may utilize a software defined network (SDN). SDN and a simple IP may be based, at least in part, on user equipment, that provide a wireless management and control framework that enables common wireless management and control, such as mobility management, radio resource management, QoS, load balancing, etc., across many wireless technologies, e.g. LTE, Wi-Fi, and future 5G access technologies; decoupling the mobility control from data planes to let them evolve and scale independently; reducing network state maintained in the network based on user equipment types to reduce network cost and allow massive scale; shortening cycle time and improving network upgradability; flexibility in creating end-to-end services based on types of user equipment and applications, thus improve customer experience; or improving user equipment power efficiency and battery life—especially for simple M2M devices—through enhanced wireless management.

While examples of a system in which alerts for securing biological devices can be processed and managed have been described in connection with various computing devices/processors, the underlying concepts may be applied to any computing device, processor, or system capable of facilitating a telecommunications system. The various techniques described herein may be implemented in connection with hardware or software or, where appropriate, with a combination of both. Thus, the methods and devices may take the form of program code (i.e., instructions) embodied in concrete, tangible, storage media having a concrete, tangible, physical structure. Examples of tangible storage media include floppy diskettes, CD-ROMs, DVDs, hard drives, or any other tangible machine-readable storage medium (computer-readable storage medium). Thus, a computer-readable storage medium is not a signal. A computer-readable storage medium is not a transient signal. Further, a computer-readable storage medium is not a propagating signal. A computer-readable storage medium as described herein is an article of manufacture. When the program code is loaded into and executed by a machine, such as a computer, the machine becomes a device for telecommunications. In the case of program code execution on programmable computers, the computing device will generally include a processor, a storage medium readable by the processor (including volatile or nonvolatile memory or storage elements), at least one input device, and at least one output device. The program(s) can be implemented in assembly or machine language, if desired. The language can be a compiled or interpreted language, and may be combined with hardware implementations.

The methods and devices associated with a telecommunications system as described herein also may be practiced via communications embodied in the form of program code that is transmitted over some transmission medium, such as over electrical wiring or cabling, through fiber optics, or via any other form of transmission, wherein, when the program code is received and loaded into and executed by a machine, such as an EPROM, a gate array, a programmable logic device (PLD), a client computer, or the like, the machine becomes a device for implementing telecommunications as described herein. When implemented on a general-purpose processor, the program code combines with the processor to provide a unique device that operates to invoke the functionality of a telecommunications system.

While the disclosed systems have been described in connection with the various examples of the various figures, it is to be understood that other similar implementations may be used or modifications and additions may be made to the described examples of a telecommunications system without deviating therefrom. For example, one skilled in the art will recognize that a telecommunications system as described in the instant application may apply to any environment, whether wired or wireless, and may be applied to any number of such devices connected via a communications network and interacting across the network. Therefore, the disclosed systems as described herein should not be limited to any single example, but rather should be construed in breadth and scope in accordance with the appended claims.

In describing preferred methods, systems, or apparatuses of the subject matter of the present disclosure—secure biological devices or associated systems—as illustrated in the Figures, specific terminology is employed for the sake of clarity. The claimed subject matter, however, is not intended to be limited to the specific terminology so selected. In addition, the use of the word “or” is generally used inclusively unless otherwise provided herein.

This written description uses examples to enable any person skilled in the art to practice the claimed subject matter, including making and using any devices or systems and performing any incorporated methods. Other variations of the examples are contemplated herein.

Methods, systems, and apparatuses, among other things, as described herein may provide for securing or authenticating a biological device or remote device communicatively connected thereto. A method, system, computer readable storage medium, or apparatus provides for receiving bio related information from a biological device; receiving other information; storing the bio related information and the other information; storing statistical information associated with the bio related information or the other information; generating (or otherwise receiving) a threshold for a period associated with the bio related information and the other information (or the statistical information); sending a first request, wherein the first request is for access to bio related information or the other information of the biological device or control of the biological device; in response to the first request, receiving a first message, wherein the first message comprises a first indication of current bio related information for the biological device or historical bio related information for the biological device; or based on the first message, authenticating the biological device, wherein the authenticating is a based on a comparison of the first indication from the biological device with the threshold. All combinations in this paragraph and the following paragraphs (including the removal or addition of steps) are contemplated in a manner that is consistent with the other portions of the detailed description.

A method, system, computer readable storage medium, or apparatus provides for receiving bio related information from a biological device; receiving other information; storing the bio related information and the other information; receiving a first request, wherein the first request is for access to bio related information or the other information of the biological device or control of the biological device; in response to the first request, sending a first message, wherein the first message comprises a request for a first indication of statistical bio related information for the biological device or historical bio related information for the biological device; receiving a first response to the first request; or based on the first response, authenticating the remote device, wherein the authenticating is a based on a comparison of the first indication from the remote device with a threshold for a period associated with the bio related information and the other information (or the statistical information). All combinations in this paragraph (including the removal or addition of steps) are contemplated in a manner that is consistent with the other portions of the detailed description.

The method, system, computer readable storage medium, or apparatus provides for sending a request to change a first parameter or a second parameter of a biological device; based on the request, determining the crypto currency value corresponding to the change of the first parameter or the second parameter; determining whether the crypto currency value corresponding to the change is less than an available crypto currency associated with the biological device; based on the crypto currency value corresponding to the change being less than the available crypto currency associated with the biological device, debiting the available crypto currency; in response to the debiting the available crypto currency, granting access to the biological device; or sending information about the debit to multiple devices that include a distributed ledger. The authenticating may include comparing the indication from the biological device with a threshold, wherein the threshold may be based on statistical information of the bio related information or other information. The authenticating may be based on a comparison of the first indication from the remote device with a threshold for a period associated with the bio related information or the statistical information. All combinations in this paragraph or the previous paragraphs (including the removal or addition of steps) are contemplated in a manner that is consistent with the other portions of the detailed description.

What is claimed:

1. A method for authenticating a biological device, the method comprising:
sending a request, wherein the request is for access to bio related information or other information of a biological device or control of the biological device;
in response to the first request, receiving a first message, wherein the message comprises an indication of current bio related information for the biological device or historical bio related information for the biological device; and
based on the message, authenticating the biological device.

2. The method of claim 1, wherein the bio related information is at least one factor in multiple factor authentication of the biological device.

3. The method of claim 1, wherein the authenticating comprises comparing the indication from the biological device with a threshold.

4. The method of claim 1, wherein the authenticating comprises comparing the indication from the biological device with a threshold, wherein the threshold is based on statistical information of the bio related information or other information.

5. The method of claim 1, wherein the other information comprises an indication of a location of the biological device.

6. The method of claim 1, wherein the other information comprises an identifier of a wireless device nearby the biological device.

7. The method of claim 1, wherein the bio related information comprises heart rate or temperature of a person near the biological device.

8. A method for authenticating a remote device communicatively connected with a biological device, the method comprising:
receiving a first request, wherein the first request is for control of the biological device or access to bio related information or other information of the biological device;
in response to the first request, sending a message, wherein the message comprises a second request for an indication of statistical bio related information for the biological device or historical bio related information for the biological device;
receiving a response to the second request; and
based on the response, authenticating the remote device.

9. The method of claim 8, wherein the biological device is an implanted device.

10. The method of claim 8, wherein the authenticating comprises comparing the indication from the biological device with a threshold.

11. The method of claim 8, wherein the authenticating comprises comparing the indication from the biological device with a threshold, wherein the threshold is based on statistical information of the bio related information or other information.

12. The method of claim 8, wherein the other information comprises an indication of a location of the biological device.

13. The method of claim 8, wherein the other information comprises an identifier of a wireless device nearby the biological device.

14. The method of claim 8, wherein the bio related information comprises heart rate or temperature of a person near the biological device.

15. A system, the system comprising:
one or more processors; and
memory coupled with the one or more processors, the memory storing executable instructions that when executed by the one or more processors cause the one or more processors to effectuate operations comprising:
sending a request, wherein the request is for access to bio related information or other information of a biological device or control of the biological device;
in response to the first request, receiving a first message, wherein the message comprises an indication of current bio related information for the biological device or historical bio related information for the biological device; and
based on the message, authenticating the biological device.

16. The system of claim 15, wherein the bio related information is at least one factor in multiple factor authentication of the biological device.

17. The system of claim 15, wherein the authenticating comprises comparing the indication from the biological device with a threshold.

18. The system of claim 15, wherein the authenticating comprises comparing the indication from the biological device with a threshold, wherein the threshold is based on statistical information of the bio related information or other information.

19. The system of claim 15, wherein the other information comprises an indication of a location of the biological device.

20. The system of claim 15, wherein the other information comprises an identifier of a wireless device nearby the biological device.
